(19) United States
(12) Patent Application Publication
(10) Pub. No.: US 2007/0031341 A1
(43) Pub. Date: Feb. 8, 2007

(54) Methods of delivering therapeutics to the brain

(76) Inventors: Thomas M. DiMauro, Southboro, MA (US); Mohamed Attawia, Canton, MA (US); Richard Toselli, Barrington, RI (US); Sean Lilienfeld, Sharon, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/200,438

(22) Filed: Aug. 8, 2005

(51) Int. Cl. A61K 38/44; A61N 1/30; A61L 9/04; A61K 31/355; A61K 31/405; A61K 31/375; A61K 33/00

(52) U.S. Cl. 424/45; 424/600; 424/94.4; 514/419; 514/474; 514/458; 604/20

(57) ABSTRACT

Intranasal iontophoretic administration of therapeutics such as Reduced Water (RW) or lubeluzole as a means of treating diseases of the CNS involving oxidative stress.

### BACKGROUND OF THE INVENTION

Many inflammatory or immunogenic responses in the body in general and the central nervous system in particular involve the generation of reactive oxygen species (ROS). These ROS include hydroxyl radicals (OH), hydrogen peroxide (H₂O₂), superoxide ion (O₂) and singlet oxygen (O). In some situations, ROS are produced by native white blood cells such as neutrophils. In others, ROS are produced by the interaction of oxygen with metallic constituents such as iron. Although these ROS are considered to be helpful in many situations, such as in controlling infection and wound healing, there are many other situations wherein the overabundance of ROS is associated with disease states. In these disease-associated situations, the role of the ROS is known as “oxidative stress”.

There are many diseases of the central nervous system (CNS) that have a significant oxidative stress component. For example, excessive oxidative stress is thought to be the primary cause of Parkinson's Disease.

Oxidative stress is a major component in the onset and progression of Parkinson's Disease. It is believed that the substantia nigra contains high levels of iron. Iron is an important catalyst in the conversion of hydrogen peroxide and superoxide ions into the more potent hydroxyl radical.Oxidative stress is also thought to be a contributing factor to multiple sclerosis (MS).Oxidative stress is also thought to contribute to the pathogenesis of Alzheimer's Disease (AD) and stroke.

The literature describes the general use of anti-oxidants as potent neuroprotective agents for CNS diseases. These anti-oxidants include metal-based antioxidants such as catalase (CAT), superoxide dismutase (SOD), glutathione peroxidase (GSH-Px), and vitamins such as Vitamin A, C and E. Parkin. Relat. Disord., 1002, Jul. 7, (3) 243-6, describes the use of fullerene-based antioxidants as neuroprotective drugs. In addition, oral administration of ascorbic acid has been tried as a therapy for Parkinson's Disease. The well-known DATATOP study involved the oral administration of anti-oxidants to Parkinson's patients.

Despite the attractiveness of using anti-oxidants to treat oxidative stress, clinical results have been relatively disappointing. In general, these pharmacologic anti-oxidants are polar molecules and so have difficulty traversing the blood-brain barrier. It has been estimated that less than 1% of a dose of a polar therapeutic agent is able to cross the blood-brain barrier.

The concept of using iontophoresis to drive charged molecules through the cribriform plate and into the brain is well known in the art. For example, Lerner, J. Drug Targeting, Jun. 2004, 12(5) 273-280 (Lerner I) discloses the use of intranasal iontophoresis to drive charged drugs into the brain. Lerner I sought to delivered octreotide (a molecule with a charge of +2 at pH 5) from the nasal cavity to the brain, and enhanced this delivery by electrophoretic means (also called “iontophoresis”). Lerner I placed a silver anode having a octreotide reservoir at the top of the nasal cavity near the cribriform plate and placed a return cathode near the back of the head, and then applied a voltage therebetween to produce a current of about 3 mA through the brain. Lerner I reported that the amount of octreotide delivered to the brain increased about 2-13 fold when enhanced by iontophoresis when compared to controls.

### SUMMARY OF THE INVENTION

In a first aspect of the present invention, the present inventors have developed inventions related to the iontophoretic delivery of selected molecules to the brain: namely, reduced water, lubeluzole, catalase, SOD, melatonin, αMSH, and Vitamins C and E.

Therefore, in accordance with the present invention, there is providcd a method of treating a patient having a disease of the central nervous system, comprising:

a) intranasally administering through a cribriform plate an effective amount of a therapeutic agent selected from the group consisting of reduced water, lubeluzole, catalase, SOD, melatonin αMSH, and Vitamins C and E.

In a second aspect of the present invention, the present inventors have also developed novel methods of delivery of therapeutic molecules to the brain.

The present inventors have developed inventions related to the administration of Reduced Water (RW) as a means of treating diseases of the CNS.

Huang, Kidney Int'l. 64, (2003) 704-714 characterizes reduced water as the ideal scavenger of reactive oxygen species (ROS). Shirahata, Biochem. Biophys. Res. Comm. 234, (1997) 269-274 reports that reduced water scavenges active oxygen species and protects DNA from oxidative damage. Huang, Kidney Int'l. 64, (2003) 704-714, reports that reduced water ameliorated hemodialysis-induced oxidative stress in end-stage renal disease patients. Hanaoka, Biophys. Chem. 2004 Jan. 1, 107(1) 71-82 reports on the mechanism of enhanced antioxidative effects against superoxide anion radicals of reduced water produced by electrolysis.

Accordingly, delivering reduced water to a CNS that is under oxidative stress will have the effect of therapeutically removing the toxic ROS from the CNS environment.

The present inventors believe that intranasal administration of reduced water has a number of desirable characteristics. First, because it is delivered intranasally, it can be provided to the brain via a relatively non-invasive means. This non-invasive delivery allows for the chronic delivery of the reduced water, and so is of great utility in progressive diseases such as Parkinson's Disease, Multiple Sclerosis and Alzheimer's Disease. Second, the intranasal delivery of reduced water is a local delivery that delivers large amounts of the therapeutic molecule to the site in need of therapy without also delivering the molecule systemically. Third, the intranasal delivery in general has been shown to be a means for providing therapy quickly to the brain via the CSF (and so may be of great utility in treating acute conditions such as stroke). Fourth, because intranasal delivery facilitates delivery across the blood-brain barrier, there is a potential. for delivering relatively large amounts of reduced water to the brain. Fifth, because reduced water consists essentially of water having a surplus of electrons that will react with ROS, the safety of both the initial product and the reaction products is quite high.

### DESCRIPTION OF THE FIGURES

FIG. 1 is a cross-section of a device of the present invention comprising an intranasal cathodic reservoir holding reduced water.

FIGS. 2a-b disclose a hand-held intranasal cathodic probe of the present invention.

FIG. 2c shows the probe of FIG. 2a adjacent the cribriform plate.

FIG. 3 discloses a bipolar probe of the present invention having a cathodic reservoir.

FIGS. 4a-b discloses a bipolar probe of the present invention having an anodic reservoir.

FIGS. 5a-5b disclose a bipolar implant of the present invention having an anodic reservoir.

FIG. 6 discloses an iontophoretic transcranial implant of the present invention.

FIGS. 7a-7e disclose a preferred use of a novel transcranial device of the present invention.

FIGS. 8a-8b disclose preferred uses of novel transcranial systems of the present invention.

FIG. 9 discloses a novel transcranial device of the present invention having infection control features.

FIGS. 10a-b show a pressure-based delivery device of the present invention.

FIG. 11 shows the pressure-based delivery device of FIG. 10a adjacent the cribriform plate.

FIGS. 12A-C disclose another embodiment suitable for pressurized delivery through the cribriform plate, wherein the distal portion of the device is controllably expandable.

### DETAILED DESCRIPTION OF THE INVENTION

A number of theories have been proposed as to why reduced water has antioxidant properties. According to one theory, reduced water comprises activated hydrogen (which appears to be a hydrogen ion having a second electron, also called a “hydride” ion). See, e.g., Shirahata, Biochem. Biophys. Res. Comm. 234, (1997) 269-274. According to another theory, reduced water has antioxidant capabilities because it has a high activated dissolved molecular hydrogen. See, e.g., Hanaoka, J. Applied Electrochem. 31, 1307-1313, 2001. According to another theory, reduced water has antioxidant capabilities because is alters the ionic product of water. See, e.g., Hanaoka, Biophys. Chem. 2004 Jan. 1, 107(1) 71-82.

Regardless of the theory, it appears that providing reduced water to a CNS under oxidative stress will have the effect of detoxifying ROS within the CNS, thereby therapeutically lowering the oxidative stress.

Reduced water may have SOD-like activity. According to Kamanli, Cell Biochem. Func. 2004, 22:53-57, SOD catalyses dismutation of the superoxide anion into hydrogen peroxide.

It has also been proposed that reduced water has catalase-like activity. Hanaoka, J. Applied Electrochemistry, 31, 1307-1313, (2001). According to Kamanli, Cell Biochem. Func. 2004, 22:53-57, catalase detoxifies hydrogen peroxide and converts lipid hydroperoxides into non-toxic alcohols, and is essential for the inhibition of inflammation related to the function of neutrophils

“Reduced water” comprises activated atomic hydrogen and has been shown to scavenge reactive oxygen species in a manner similar to both catalase and SOD. It is very stable and potent, and has a significant negative redox potential, as it includes molecular hydrogen that has gained an electron. Reduced water contains a large amount of dissolved molecular hydrogen.

Reduced water typically has a very large negative redox potential. The large electronegativity allows reduced water to easily combine with the ROS. In some embodiments, the redox potential of reduced water is between about −50 mV and −1000 mV. Preferably, the redox potential of the reduced water is between about −250 mV and about −1000 mV. More preferably, the redox potential of the reduced water is between about −500 mV and about −1000 mV.

Reduced water typically has a high level of dissolved hydrogen. In some embodiments, the reduced water comprises between about 100 ppm and 1200 ppm dissolved hydrogen. Preferably, the reduced water comprises at least 400 ppm dissolved hydrogen, more preferably at least 600 ppm, more preferably at least 800 ppm. In contrast, natural water typically comprises less than about 10 ppm dissolved hydrogen.

Reduced water typically has a low level of dissolved oxygen. In some embodiments, the reduced water comprises less than 10 parts per million (ppm) dissolved oxygen, preferably less than 9 ppm, preferably less than 8 ppm. In contrast, typical tap water has a dissolved oxygen content of 10 ppm.

In some embodiments, reduced water has a high pH. In some embodiments, the reduced water has a pH of between 8 and 12. In some embodiments, however, the reduced water is neutralized with an acid prior to its administration. In contrast, typical tap water has a pH of about 7.5.

In preferred embodiments, reduced water can be produced by simply electrolyzing water in two different containers. Reduced water is produced at the cathode of the electrolytic cell. U.S. Pat. Nos. 6,475,371 (Shirahata) and 6,585,868 (Chihara), the specifications of which are incorporated by reference herein in heir entireties, each disclose methods of making reduced water.

In some embodiments, an electrolyte is added to water in order to enhance the conductivity of the water during electrolysis. In some embodiments, thereof, the added electrolyte is a salt, preferably NaCl. In other embodiments, the electrolyte is a base comprising OH, and is preferably NaOH.

According to Marranness, J. Pharmacol. Exp. Therapeutics, 295(2) 2000, 531-545, lubeluzole is the (+)-S enantiomer of a benzothiazole derivative (See FIG. _) that has a neuroprotective action in animal models of focal and global ischemia, in which it reduces sensormotor deficits and the infarct volume. Lubeluzole inhibits glutamateinduced nitric oxide related neurotoxicity and blocks neurotoxicity induced by nitric oxide donors. Because of these qualities, lubeluzole has been proposed as a therapeutic in early stage ischemic stroke. However, the delivery of lubcluzole across the blood brain barrier has been found to be problematic.

Because lubeluzole has a PKa of 7.6 it is present in its protonated cationic form at pHs less than 7.6. For example, at pHs of 7.4 and 6.0, about 61% and 97% of lubeluzole is respectively present in its protonated cationic form. Therefore, the skilled artisan can expect a significant amount of lubeluzole to be present in its cationic form at relatively mild pHs and particularly at the pHs of physiologic CSF (˜7.5). Moreover, support for the transport of benzothiazole derivatives is found in the literature. Levamisole is believed to be a benzothiazole derivativethat has been transdermally transported iontophoretically in order to treat herpes simplex virus and recurrent aphthous stomatitis.

In sum, since lubeluzole will be present substantially in its cationic form at mild pHs, has a very low molecule weight, and has a benzothiazole structure that has been shown to be amenable to iontophoretic transport, it appears to be a suitable candidate for iontophoretic transport into the brain.

In some embodiments, melatonin is used as the therapeutic agent. The iontophoretic transport of melatonin has been reported in the literature. Escames, J. Neuroendocrinology, 2004, 16, 929-935. Melatonin is able to cross the blood brain barrier. Gupta, Indian J Physiol Pharmacol. 2003 October;47(4):373-86 Lastly, melatonin is a very effective antioxidant and neuroprotective agent. Escames, J. Neuroendocrinology, 2004, 16, 929-935 reports that melatonin counteracts brain oxidative damage in NMDA models of excitotoxicity.

In some embodiments, the therapeutic agent comprises Vitamin C. As a watersoluble antioxidant, vitamin C scavenges aqueous peroxyl radicals that participate in the lipid degradation process. It works along with vitamin E, a fat-soluble antioxidant, and glutathione peroxidase to stop free radical chain reactions. As an antioxidant, vitamin C's primary role is to neutralize free radicals. Since ascorbic acid is water soluble, it can work both inside and outside the cells to prevent free radical damage. Free radicals will seek out an electron to regain their stability. Vitamin C is an excellent source of electrons; therefore, it can donate electrons to free radicals such as hydroxyl and superoxide radicals and quench their reactivity. Vitamin C also works along with glutathione peroxidase to revitalize vitamin E, a fat-soluble antioxidant. In addition to its work as a direct scavenger of free radicals in fluids, then, vitamin C also contributes to the antioxidant activity in the lipids. The iontophoretic transport of Vitamin C has been reported in the literature. Ebihara, J. Dermatological Science, (2003) 32, 217-222. It is believed that as little as 10 μM Vitamin C is an effective anti-inflammatory concentration. Accordingly, the formulation comprising an effective amount of Vitamin C comprises at least 100 μM, more preferably at least 250 μM, more preferably at least 500 μM Vitamin C. Vitamin C is defined to include ascorbic acid and its derivatives.

In some embodiments, the therapeutic agent comprises Vitamin E.It is believed that.Vitamin E would also be a superior anti-oxidant support for grafted cells. Gonzalez, Cell Biology Int'l, 28(2004) 373-80, clearly reports the effectiveness of Vitamin E in treating PD cells in culture. In particular, Gonzalez reports that 100 μM Vitamin E increased the in vitro viability of cerebellar granule cells exposed to MPP+ neurotoxin from about 50% to about 85%. Vitamin E appeared to be the most protective anti-oxidant tested by Gonzalez. Therefore, in some embodiments, the Vitamin E concentration of between about 10 and 1000 μM

In some embodiments, catalase is used as the therapeutic agent. Gonzalez, Cell Biology Int'l, 28(2004) 373-80, clearly reports the effectiveness of catalase in enhancing the viability of PD cells in culture. In particular, Gonzalez reports that 50 U/ml catalase increased the in vitro viability of cerebellar granule cells exposed to MPP+ neurotoxin from about 50% to about 75%. Furthermore, it is believed that catalase is superior to the anti-oxidants recited in the prior art because it is not only an anti-oxidant, it is also neurotrophic towards CNS neurons. See Wallicke, J. Neuroscience, Apr. 1986, 6(4), 1114-21. Human erythrocyte catalase (Cat. No. A50136H) is available from Biodesign International, Saco, Me.

In some embodiments, SOD is used as the therapeutic agent. SOD has been shown to be neuroprotective in a rat model of Parkinson's disease. Nakao Nat. Med. 1995, Mar 1, (3) 226-231, reports that the survival of grafted dopaminergic neurons in transgenic rats designed to overexpress Cu/Zn SOD was about four times higher than those in control rats, and there was also a similar increase in functional recovery.

In some embodiments, alpha-melanocyte stimulating hormone (αMSH) is used as the therapeutic agent. Alpha-melanocyte stimulating hormone (αMSH) is a hormone produced mainly in the pituitary gland and functions as a control of skin pigmentation. This molecule, produced by post-translational processing of pro-opiomelanocortin (POMC), is a 13 amino acid peptide highly conserved across phylogeny and widely expressed in tissues. Eberle A N, The Melanotropins, Basel (ed. S. Karger) 1988. The peptide is produced by the pituitary and by many extrapituitary cells, including monocytes, astrocytes, gastrointestinal cells, and keratinocytes. Endogenous αMSH modulates fever and inflammation. αMSH also has anti-pyretic qualities and is released during fever. Tatro, Clin. Infect. Dis. 2000, 31: S190-201. The literature has further reported that αMSH can be used to quell an IL-1β-induced fever. Accordingly, delivery of αMSH may be therapeutic for patients suffering from a stroke.

aMSH has been extensively linked to immunosuppression and tolerance. Lipton, News Phys. Sci., 15, 2000, 192-5 reports that aMSH is an important neuroimmunomodulator. Luger, NY Acad. Sci. 1999, Oct. 20, 885, 209-16 reports that aMSH plays an important role in light-mediated immunosuprression. The literature has extensively reported that αMSH upregulates anti-inflammatory processes. For example, Luger, Ann. NY Acad. Sci., 2003 June 994:133-40 reports that a αMSH modulates antigen presenting function and upregulates IL-10, a known anti-inflammatory cytokine. Taylor, Immunol. Cell Biol., 2001 August 79(4) 358-67 reports that αMSH induces the production a TGF-B, a known anti-inflammatory cytokine, by T cells. The literature has also extensively reported that αMSH antagonizes the activity of pro-inflammatory molecules, and it is believed that αMSH is involved in the regulation of the NFκB pathway of pro-inflammatory cytokine production. Luger ,Pathobiology, 1999, 67(5-6), 318-21 reports that αMSH functions to downregulate NFkB activity, thereby downregulating pro-inflammatory cytokine production. Luger concludes that αMSH plays an essential role in inducing tolerance. The inhibition of IL-1B-induced acute inflammation by MSH analogs has also been reported in the literature. See Hiltz, Cytokine, 1992, July 4 (4): 320-8; Ceriani, Neuroendocrinology, 1994, February 59(2):138-43; and Watanabe, Brain. Bull. Res., 1993, 32(3):311-4. Wong, Neuroimmunomodulation, 1997 January -February 4(1), 37-41 reports the modulation of TNF-a production within an inflamed brain by MSH, and concludes that aMSH represents a potential local mechanism of anti-inflammatory action within the brain. MSH inhibits the inflammatory cascade at many sites: it reduces production of NO (Star, Proc Natl Acad Sci USA1995; 92:8016-8020), proinflammatory cytokines (Lipton, Immunol Today 1997; 18:140-145), monocyte chemoattractant protein 1 (MCP-1), and interleukin 8 (IL-8), and markedly decreases neutrophil chemotaxis in vivo and in vitro (Mason,. J Immunol 1989; 142:1646-1651; Catania, Peptides 1996; 17:675-679. These effects of the peptide are exerted, at least in part, through inhibition of activation of the nuclear factor NF-.kappa.B, a pivotal transcription factor for genes that encode proinflammatory cytokines, chemokines, and adhesion molecules. Manna, J Immunol 1998; 161:2873-2880; Ichiyama, Exp Neurol 1999; 157:359-365. See also a review in Lipton, “Marshalling the Anti-Inflammatory Influence of the Neuroimmunomodulator MSH”, News Physiol. Sci., Vol. 15, August 2000, pp. 192-195. Lipton states that MSH inhibits TNF-a and may limit neurodegeneration and other CNS disorders that have an inflammatory component.

The literature has further appreciated the potential therapeutic possibilities of cerebrally administering αMSH in order to quell damaging brain-related inflammatory processes. Ichiyama, Brain. Res., 1999, Jul 31, 836 (102) 31-7, reports that systemic administration of aMSH inhibits brain inflammation, and does so by inhibiting the NFKB pathway. Rajora, J. Neurosci., 19997, Mar 15, 17(6) 2181-6 reports that αMSH inhibits brain inflammation by modulating TNF-a. Galimberti, Biochem. Biophys. Res. Comm., 1999 Sept. 16, 263(1) 251-6 examined a possible role for MSH in mediating Alzheimer's Disease and reported that aMSH inhibits NO and TNFa by microglia activated with BAP.

Because αMSH is cationic at physiologic pHs (Biaggi, Eur. Biophys. J., 1996, 24(4) 251-9 and is a small molecule, it is likely to be amenable to iontophoretic delivery to the brain.

Because each of reduced water, lubeluzole, melatonin and Vitamin C carries a significant charge and has a low molecular weight, it is believed that the intranasal delivery of these compounds may be significantly enhanced by iontophoresis.

In preferred embodiments, the cathode is placed near the top of the nasal cavity so that it abuts the cribiform plate. This abutment places the cathode in electrical connection with the tissue of the olfactory bulb and therefore the brain. Preferably, a pair of cathodes are placed bilaterally in each half of the nasal cavity. However, in some embodiments, a single cathode may be placed in a single half of the nasal cavity.

In some embodiments, the length of the cathode spans at least one-quarter of the length of the cribriform plate. Because the length of the cribriform plate of the typical adult is at least about 2 cm, the length of the cathode should be at least about 2 cm. More preferably, the length of the cathode spans at least one-half of the length of the cribriform plate, more preferably substantially all of the cribriform plate. Full coverage of the cribriform plate is advantageous because it provides a larger cross-section through which the reduced water may pass, thereby offering a lower resistance path.

In some embodiments, the cathode is made of a conformable material that allows the anode to make good electrical contact with the nasal mucosa while avoiding damage to the nasal mucosa. In preferred embodiments thereof, the cathode comprises a wool portion. This wool is advantageous because it not only conforms to the nasal mucosa abutting the cribriform plate, it also conforms to the lateral conchae provides a snug fit within the nasal cavity, thereby preventing fallout.

The cathode may be made from any conventional conductive material used in biomedical applications, including silver and. copper. In some embodiments, the cathode comprises magnesium. A magnesium cathode possesses special advantage in that it will beneficially corrode and thereby provide magnesium ions to the brain tissue. It has been reported that magnesium ions impede the deposition of BAP, a neurotoxic compound associated with AD.

Preferred devices for delivering the above-mentioned molecules to the brain will now be discussed. In most cases, the devices are designed for delivering anionic reduced water, and so will be designed with cathodic reservoirs. However, it should be understood that when the skilled artisan desires to deliver cationic molecules, such as lubeluzole, the polarity of the system will be reversed.

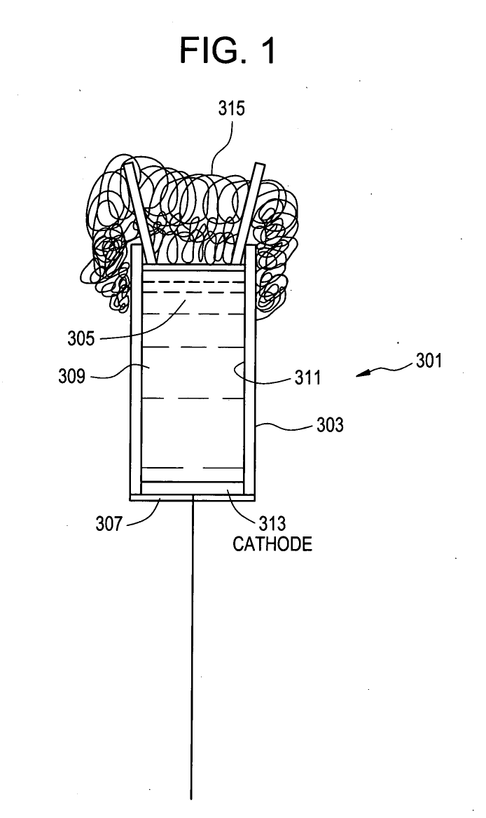

Now referring to FIG. 1, in some embodiments, there is provided a cathode 301 of the present invention, comprising:
a) a container 303 having an open distal end 305 and a closed proximal end 307, the container forming a reservoir 309 having an inner proximal face 311,
b) a cathode 313 attached to the inner proximal face of the container,
c) a porous fibrous plug 315 attached to the open distal end of the container, and
d) reduced water contained within the container and wetting porous fibrous plug.
   In practice, the porous fibrous plug is placed against the nasal mucosa adjacent the cribriform plate. When a voltage is applied to the device between the cathode and anode (not shown), the excess electrons in the reduced water is driven away from the cathode and through the cribriform plate.

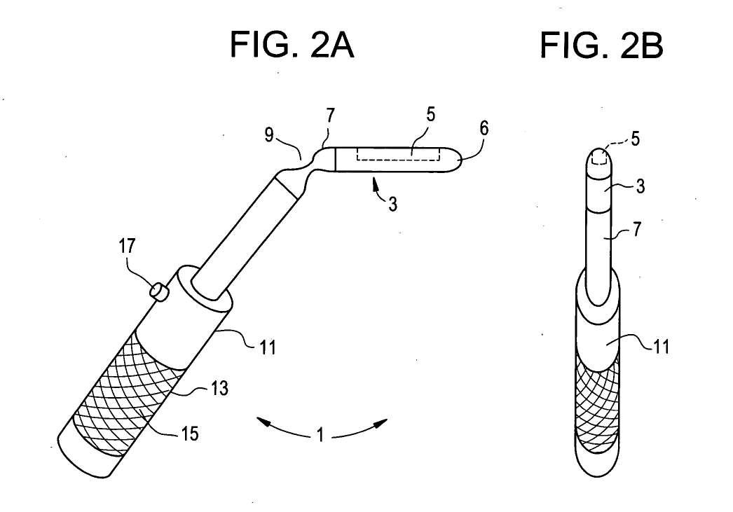

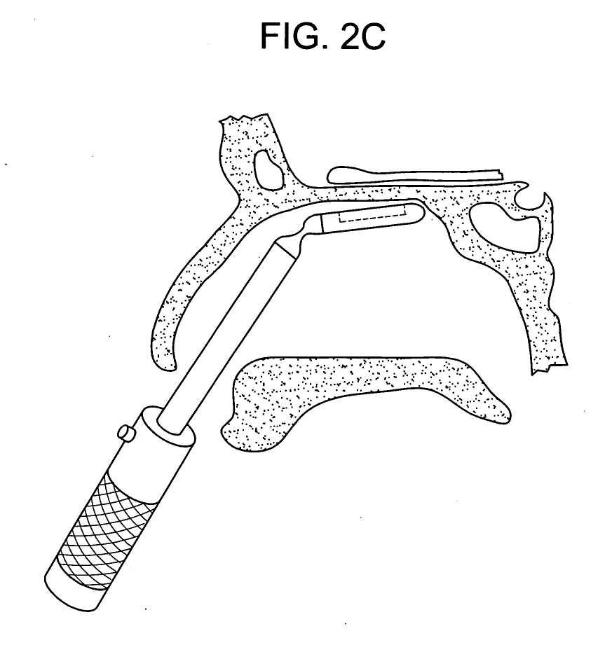

Now referring to FIGS. 2a-2c, there is provided a hand-held intranasal cathodic probe for treating a neurodegenerative disease in a patient, comprising:
a) a distal portion 3 adapted to fit within an upper portion of a nasal cavity and having a cathode 6 and a reservoir 5 adapted to contain a therapeutic agent (such as reduced water) and oriented towards the cribriform plate,
b) a flexible intermediate portion 7 having an angled, narrowed portion 9,
c) a proximal portion 11 having a handgrip 13 having a knurled surface 15 and a voltage source activation button 17,
d) a voltage source (not shown) disposed within the proximal portion and electrically connected to the cathode.

FIG. 2c shows the probe of FIG. 2a inserted within the nasal cavity and adjacent the cribriform plate.

In some embodiments, the height of the distal portion is greater than its width. This allows orientation. In some embodiments, the distal portion is detachable from the remainder of the device. This allows it to be periodically cleaned by the user. In some embodiments, the tip of the distal portion is rounded in order to ease the entry of the distal portion in the nasal passage. In some embodiments, the length of the distal portion corresponds substantially to the length of the cribriform plate. This allows the reservoir and cathode to extend along substantially the entire porosity of the cribriform plate. In some embodiments, the length of the cathode corresponds substantially to the length of the cribriform plate. In some embodiments, the cathode is oriented to face the cribriform plate upon insertion in the nasal passage. In some embodiments, the distal portion has an upper surface oriented to face the cribriform plate upon insertion.

In some embodiments, the narrowed portion is provided along only one axis, thereby providing preferred bending.

In some embodiments, the voltage source is located in the proximal portion of the device, and is also connected to an anode (not shown) through the proximal end of the device. In some embodiments, the voltage source is located in the distal portion. In some embodiments, the voltage is operated by a battery contained within the device. In some embodiments, the voltage source is provided external to the hand held device and is electrically connected to the device.

Preferably, the anode is placed so that an electric field is produced that causes reduced water to be pulled through the cribriform plate.

In some embodiments, the anode is attached to a separate probe and is preferably attached to the back of the head of the patient. When combined with a cathode situated near the cribriform plate and a voltage is applied, an electric field traversing the entire cerebral cortex is created. Accordingly, this embodiment allows the delivery of reduced water to the entire cerebral cortex.

In some embodiments, the anode is placed in the frontal sinus. When combined with a cathode situated near the cribriform plate and a voltage is applied, an electric field traversing the frontal portion of the prefrontal cortex is created. Accordingly, this embodiment allows the delivery of reduced water to the frontal portion of the prefrontal cortex.

In some embodiments, the anode is placed on the forehead of the patient. When combined with an anode situated near the cribriform plate and a voltage is applied, an electric field traversing the frontal lobe is created. Accordingly, this embodiment allows the delivery of reduced water to the frontal lobe.

In some embodiments, the anode is placed in the sphenoidal sinus. When combined with an anode situated near the cribriform plate and a voltage is applied, an electric field traversing the hind portion of the prefrontal cortex is created. Accordingly, this embodiment allows the delivery of reduced water to the hind portion of the prefrontal cortex.

The anode may be made from any conventional conductive material used in biomedical applications, including silver and copper.

In some embodiments, the devices disclosed in U.S. Pat. No. 6,410,046 “Administering Pharmaceuticals to the Mammalian Central Nervous System” (“Lerner I”); U.S. Pat. No. 6,678,553, “Device for Enhanced Delivery of Biologically Active Substances and Compounds in an organism” (“Lerner II”); US Published Patent Application No. US 2002/0183683 “Methods and Apparatus For Enhanced and Controlled Delivery of a Biologically Active Agent into the Central Nervous System of a Mammal” (Lerner IV″), and Lerner, J. Drug Testing, June 2004, 12(5) 273-280 (“Lerner V), the specifications of which are incorporated by reference in their entireties, are selected as the cathodes, anodes and power sources. In these embodiments, the reduced water is placed within the cotton balls disclosed in Lerner.

As noted above, Lerner V reported on the iontophoretic delivery of charged molecules into the brain. However, Lerner V further reported that one of the test rabbits experienced shivering and rhythmic movement during the test. The present inventors observe that Lerner used a monopolar electrode system.

Therefore, in some highly preferred embodiments of the present invention, the invention comprises a bipolar probe having both an anode and a cathode.

Without wishing to be tied to a theory, it is believed that the real benefit of iontophoresis for intranasal delivery lies in its ability to move charged molecules from the nonpolar nasal mucosa across the cribriform plate and into the CSF. Once the molecule has reached the CSF, it is readily distributed through the brain by convection. Therefore, the electric field produced by the anode and cathode need only be effective in the vicinity of the cribriform plate. If this is true, then a bipolar electrode configuration should suffice the transport needs of the present invention.

The present inventors note that Lerner V suggested that the reason for the quick delivery of octreotide throughout the brain lies in the ability of the CSF to transport the molecule.

Moreover, it is further believed that bipolar electrode configurations offer a safety advantage over the monopolar designs disclosed in Lerner I-IV. With monopolar designs, the path of the electric current between the anode and cathode is substantially uncontrolled, as it takes the path of least resistance over a large distance. This may be the reason for the shivering effect reported by Lerner. In contrast, with the bipolar design, the path of the electric current is very well controlled. Moreover, the path taken by the electric current between the anode and cathode in the bipolar design is much shorter. Accordingly, there is less resistance offered by the brain tissue and so a lower voltage may desirably be used.

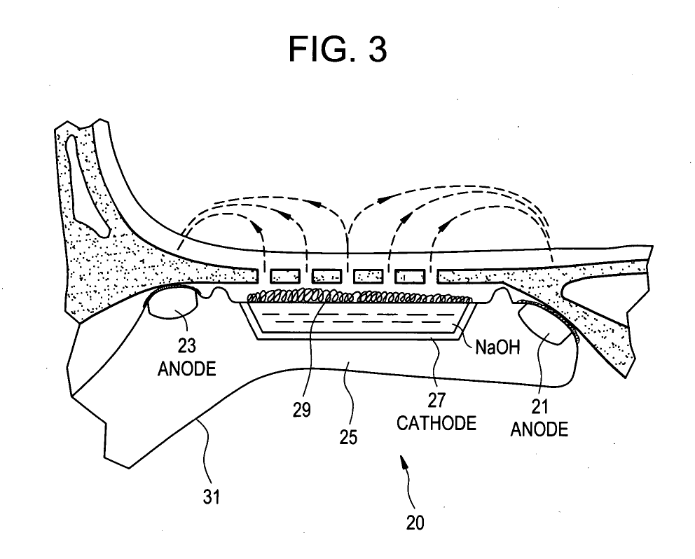

Now referring to FIG. 3, there is provided a hand-held intranasal bipolar probe for treating a neurodegenerative disease in a patient, comprising:
a) a distal portion 20 adapted to fit within an upper portion of a nasal cavity, the distal portion comprising:
   i) a distal anode 21,
   ii) a proximal anode 23, and
   iii) an intermediate portion 25 comprising a cathode 27 forming a reservoir having reduced water therein and a fibrous plug 29 overlying the reservoir and wetted by the reduced water,
b) a intermediate portion 31.

In practice, the porous fibrous plug is placed against the nasal mucosa adjacent the cribriform plate. When a voltage is applied to the device between the cathode and anode, the resulting electric field operates to drive activated hydrogen in the reduced water away from the cathode, through the porosity of the cribriform plate, and into the cerebrospinal fluid (CSF). Because the distal portion of the electric field produced in this embodiment extends posterior to the cribriform plate and into relatively nonporous bone, the reduced water will remain within the CSF. From there, it may be conveniently distributed by convection throughout the remainder of the brain.

As shown, the bipolar nature of the device will confine the electric current to a small area around the olfactory bulb. The controlled nature of the bipolar electrode provides for higher safety.

In preferred embodiments having a plurality of anodes (as shown), the current will be of a lower density as it reaches a plurality of cathodes, thereby reducing the possibility of tissue damage.

In some embodiments thereof, the distal anode is placed at the distal end of the probe so that it rests against the sphenoidal sinus portion of the nasal cavity. The resulting electric field may extend about 2 cm into the prefrontal cortex.

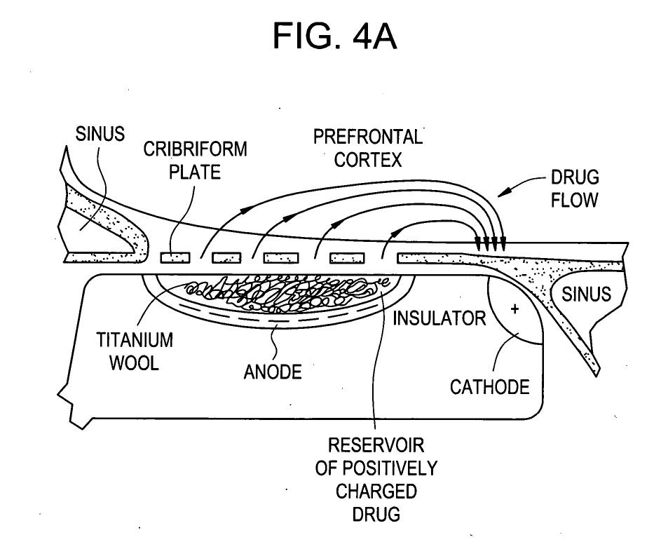

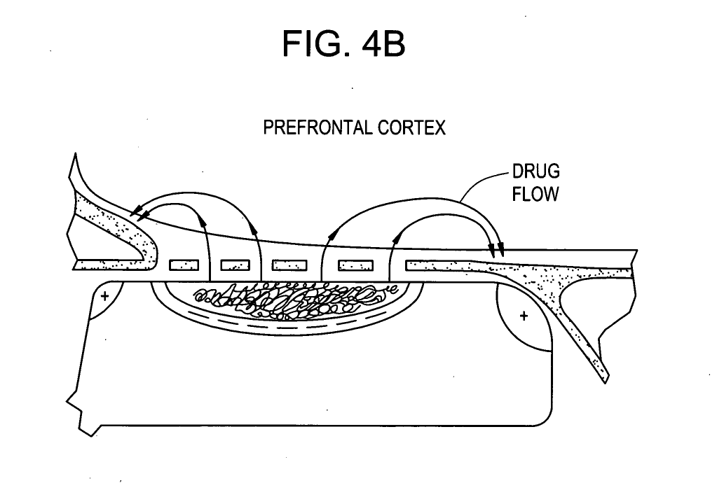

It is believed that the bipolar device of the present invention is the first bipolar intranasal device adapted to drive molecules into the brain. Accordingly, and as shown in FIGS. 4a and 4b, the bipolar device of the present invention may also be adapted to drive charged cationic molecules (such as lubeluzole and levadopa) through the cribriform plate as well.

In some situations, it may be desireable to intranasally deliver therapeutic molecules to the brain on a sustained, chronic basis. In such situations, an implantable device may be desirable. Therefore, in some embodiments, there is provided a device for providing sustained delivery of a therapeutic agent into a cribriform plate, for example, a device comprising:
a) a chamber for housing a therapeutic agent,
b) an exit port in fluid communication with the chamber,
c) an effective amount of the therapeutic agent housed within the chamber, and
d) means (such as an osmotic engine) for expelling the therapeutic agent from the chamber through the exit port.

In some embodiments, the device comprises a formulation (e.g., a first formulation) comprising an effective amount of the therapeutic agent housed within the chamber.

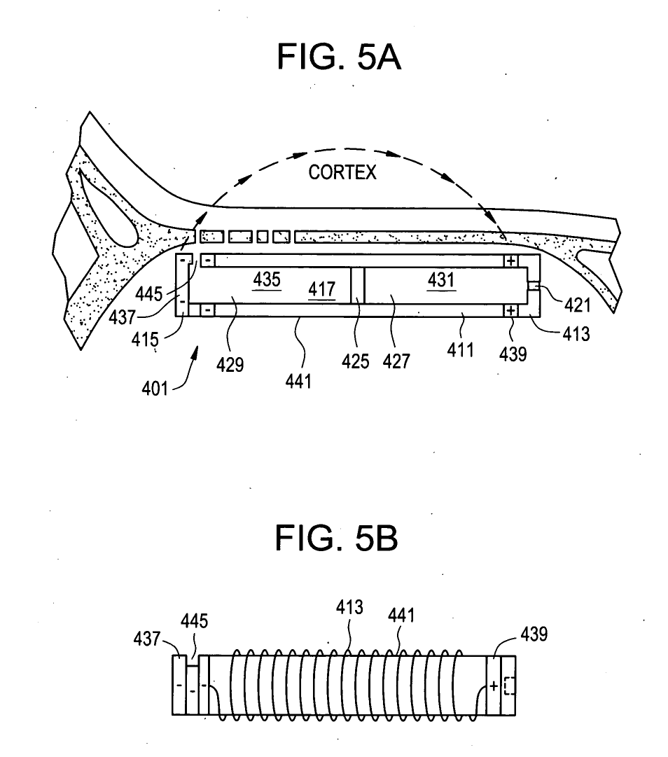

Now referring to FIG. 5aand 5b, there is provided an osmotic pump implant 1 for providing sustained delivery of a therapeutic agent into a bone. In this embodiment, the osmotic pump implant comprises:
a) a tubular member 411 including a proximal end portion 413, a distal end portion 415 and a throughbore 417 forming an exit port 445, and an outer surface 441,
b) a semi-permeable membrane 421 located in the proximal end portion of the tubular member,
c) a piston 425 provided in the tubular member, defining a proximal chamber 427 and a distal chamber 429,
d) an osmotic engine 431 located in the proximal chamber, and
e) a charged therapeutic drug 435 (such as Levadopa or lubeluzole) located in the distal chamber,
f) an antenna 413 formed around the outer surface of the tubular member,
g) a negative electrode 437 attached to the antenna and formed on the distal end portion of the outer surface of the tubular member, and
h) a positive electrode 439 attached to the antenna and formed on the proximal end portion of the outer surface of the tubular member,

The device shown in FIG. 5a works upon the following principle. Water infiltrates the semi-permeable membrane and is imbibed in the osmotic engine. Upon the receipt of water, the material selected for the osmotic engine swells. Since the semi-permable membrane is fixed and the piston is axially movable, the force produced by the swelling of the osmotic engine forces the piston to slide distally. This movement in turn forces the charged drug out the distal exit port 5. In some embodiments, design features of the device are adopted from U.S. Pat. No. 5,728,396 (“Peery”), the specification of which is incorporated by reference in its entirety.

Now referring to FIG. 5b, once an amount of the charged drug (in this case, cationic levadopa) is released in the vicinity of the cribriform plate, the antenna may be activated to provide an active anode and electrode, thereby setting up a current flow through the cribriform plate. This current will draw the release charged drug through the cribriform plate. Once the drug is pushed across the cribriform plate, it will be easily carried by the CSF flow to all parts of the brain. This can be used to transport levadopa to the brain.

It has further been reported that the intact dura is amenable to iontophoretic transport of drugs. For example, Glassenberg, (www. anestech.org/ Publications /Annual 2002/Glassenberg.html), report the iontophoretic transport of lidocaine from the epidural space across the dura and into the sub-arachnoid space. US Published Patent Application, 2004/0064127 (Lerner II) reports iontophoretically transporting a biologically active agent from the epidural space through the dura mater and into the subarachnoid space. Lerner II appears to confine this activity to the spinal area.

Therefore, in some embodiments, there is provided a method of delivering charged molecules to a patient having skull, a dura, and a subarachnoid space, comprising:
a) removing a portion of the skull to expose the dura,
b) iontophoretically delivering a therapeutic molecule through the exposed dura and into the subarachnoid space.

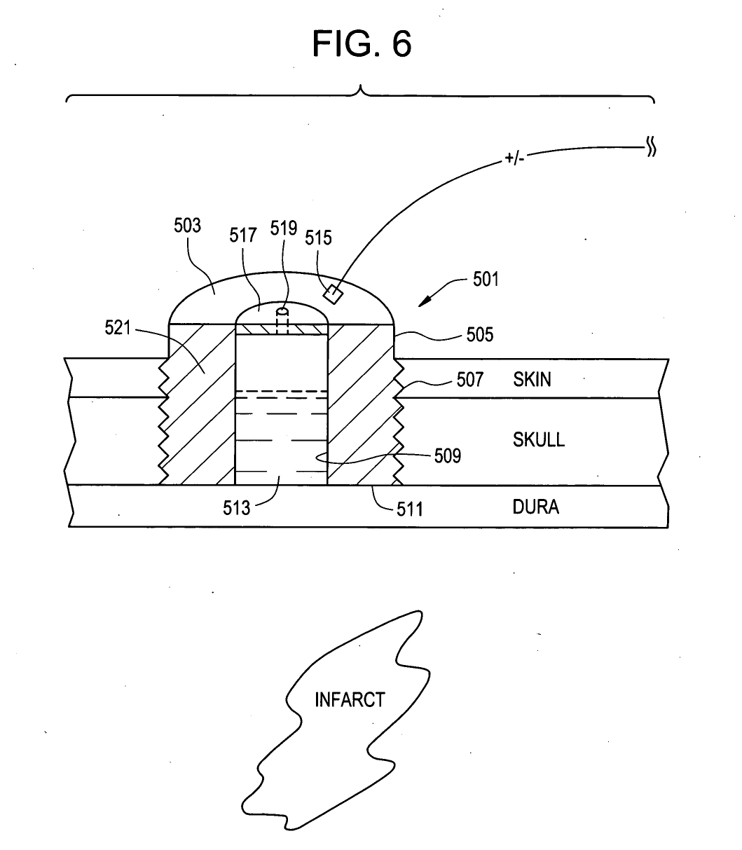

In some embodiments, and now referring to FIG. 6, there is provided a device 501 for iontophoretically delivering therapeutic molecules to the brain, comprising;
a) an upper surface 503,
b) a lateral surface 505 having a helical thread 507 adapted for screwing into bone,
c) an inner surface 509,
d) a bottom surface 511,
e) a throughbore 513 extending from the outer surface to the inner surface and defining a reservoir, and
f) an electrode 515 disposed upon the insert.

This particular device of FIG. 6 also includes a cap 517 that substantially closes off the throughbore at the upper surface of the device. The cap has an injection port 519 for injection of the therapeutic of choice into the reservoir. The bulk 521 of the device between the inner and outer surfaces is made of an electrically conductive material such as titanium so that when a voltage applied to the electrode, the bottom surface of the device effectively becomes an electrode. If desired a similar cap having an ejection port can be provided at the bottom surface of the device in order to more securely control the efflux of the therapeutic agent.

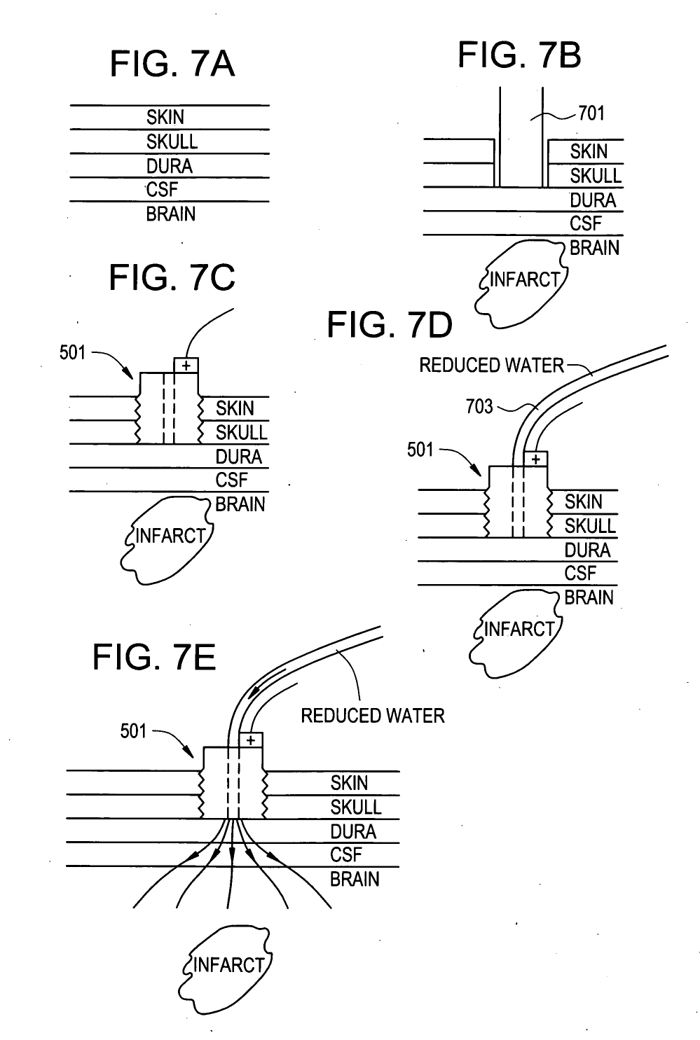

Now referring to FIGS. 7a-7e, there is provided a preferred method of preparing the cranium for the iontophoretic delivery. IN FIG. 7a, there is a cross-section of a portion of a simplified physiologic cranium, comprising, an outer scalp, a skull, a dura layer, CSF and finally brain tissue. In FIG. 7b, the skin and skull portions of the cranium are removed by a cutter 701 (such as an ultrasonic cutter) to form a hole that exposes the dura. In some embodiments, conventional duraguards may be used in order to insure the protection of the intact dura. In FIG. 7c, the device 501 of the present invention is threaded into the hole. In FIG. 7d, a delivery tube 703 is inserted into the throughbore through the outer surface of the device. In FIG. 7e, a liquid carrying the therapeutic ionic molecule (in this case, reduced water) is provided to the device through the delivery tube, and a voltage is applied that activates the cathode. The anionic species within the liquid are then repelled from the cathode and thereby pushed across the dura and into the CSF and brain tissue towards the infarct.

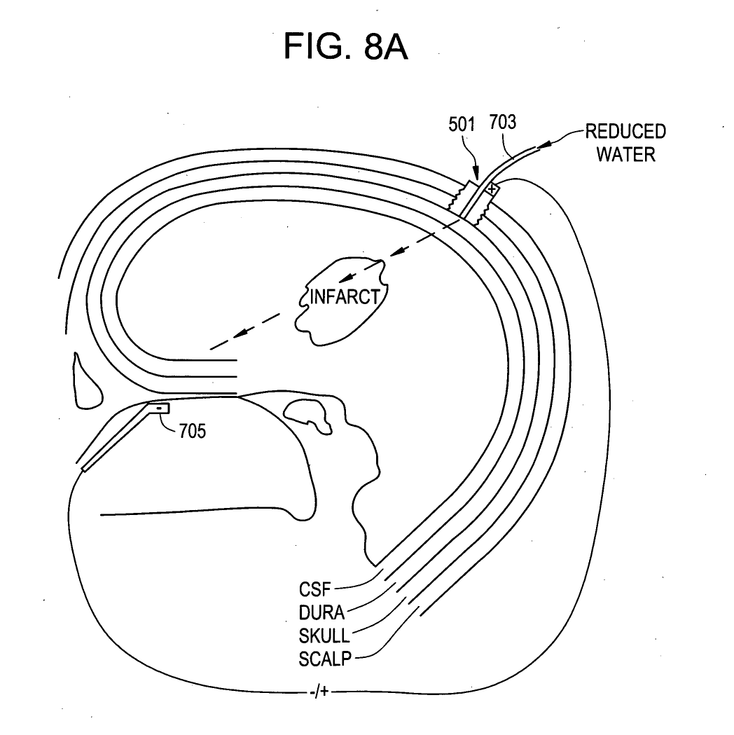

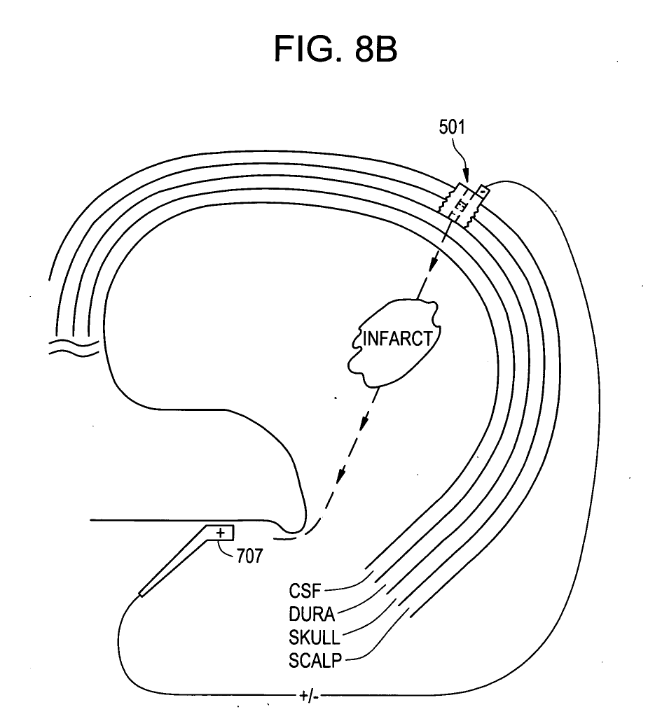

In use, and now referring to FIG. 8a, a tube 703 carrying a liquid having an ionic therapeutic molecule, such as reduced water, is placed within the throughbore of the device 501, which in this case, is connected to a voltage source and is adapted to be a cathode. The reduced water is provided to the throughbore through a delivery tube that extends through the throughbore. Concurrently, an anode 705 is placed in the patientr's nasal cavity adjacent the cribriform plate. When the voltage source is activated to apply a voltage between the electrodes, an electric field is produced between the anode and cathode that runs directly across the infarct. Because the reduced water possesses anionic activated hydrogen, these activated hydrogen species will be repelled by the local cathode and attracted to the distant anode. Accordingly, the activated hydrogen will travel across the dura, into the CSF, and up to the and into the region of the infarct. Once the activated hydrogen reaches the infarct, it will react with reactive oxygen species that have accumulated within the infarct, thereby neutralizing the ROS. Of course, this embodiment can also be practiced with cationic therapeutic agents such as lubeluzole, provided the polarity of the voltage source is reversed.

FIG. 8b represents a variation of the general system disclosed in FIG. 8a, wherein the device contained a capped reservoir of a cationic therapeutic agent, the polarity of the voltage'source is reversed, and the anode 707 is placed against the soft palate of the oronasopharyngeal cavity.

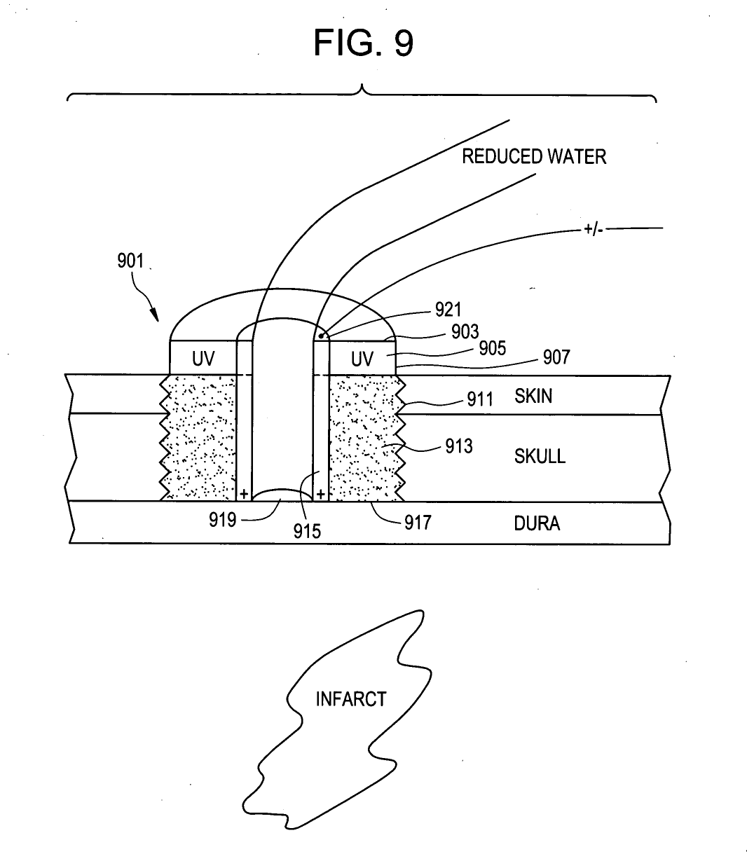

Now referring to FIG. 9, in some embodiments, the bulk material located between the upper and lower surfaces of the device comprises a UV transparent material such as silica. When such as UV transparent material is selected as the bulk material for the device, the bulk can be loaded (or the outer surface can be coated) with a photocatalytically active material such as titanium dioxide (TiO₂). TiO₂ particles are shown as being embedded in the bulk of the UV transparent device in FIG. 9. When the UV component located upon the upper surface of the device is activated, the TiO₂ becomes photocatalytically activated and TiO₂ surfaces contacting water form reactive oxygen species. These reactive oxygen species will be present only in the vicinity of the device and will provide a significant anti-bacterial function.

Further details of enabling medical device surfaces for providing photocatalytic infection control can be found in U.S. patent application Ser. No. 10/774,105 “Implant Having a Photocatalytic Unit”, filed Feb. 6, 2004 (“DiMauro et al.) (Attorney Docket No. D0601-700519) (DEP5229), the specification of which is hereby incorporated by reference in its entirety.

Therefore, in some embodiments, and now referring to FIG. 9, there is provided a device 901 for iontophoretically delivering therapeutic molecules to the brain, comprising;
a) an upper surface 903 comprising a UV light source 905,
b) a lateral surface 907 (preferably having a helical thread 911 adapted for screwing into bone and) comprising a photocatalytic material 913,
c) an inner surface 915 comprising an electrically conductive material,
d) a bottom surface 917,
e) a throughbore 919 extending from the outer surface to the inner surface and defining a reservoir, and
f) an electrode 921 disposed upon the device.
   If desired, the bottom surface can comprises either the electrically conductive material (in order to better iontophoretically direct the therapeutic agent) or the photocatalytic material (in order to provide direct infection control upon the dura). In FIG. 9, the bottom surface of the device comprises silica loaded with TiO₂ particles and so provide infection control for the dura.

In other embodiments, the implant may have an outer layer of silicone impregnated with antibiotics that slowly elute from the silicone, in a manner to the Codman BACTISEAL™ system.

The voltage applied across the electrodes should be sufficient to create the desired electric field capable of driving the ionic therapeutic species into the brain, but not so great as to cause damage to the brain. For example, in intranasal applications, the applied voltage should not be so great as to damage the olfactory bulb that lies adjacent the anode. In preferred embodiments, the applied voltage is sufficient to produce a current of between about 1 mA and about 20 mA. More preferably, the current produced thereby is less than about 10 mA.

In some embodiments, hypertonic saline is applied to the nasal mucosa or the exposed dura. This has the effect of increasingly the conductivity of the tissue surrounding the anode. In some embodiments, hypertonic saline is applied through the cribriform plate or dura and into the CSF. This has the effect of increasingly the conductivity of the CSF and extracellular fluid (ECF).

In some intranasal embodiments, an effective periodic voltage is applied so that the olfactory bulb is therapeutically depolarized. When depolarized, the olfactory bulb releases neurotrophic factors to the horizontal limb of the BB. These neurotrophic factors provide support to various portions of the limbic system and prefrontal cortex.

In some embodiments, the reservoir contains at least a second ionic species of the same polarity that also possess a therapeutic benefit.

Because the intranasal method of the present invention is non-invasive and the transdural method is minimally invasive, it is possible to repeatedly perform the procedures of the present invention. In some embodiments, the present invention is carried out at least once a month. In some embodiments, the present invention is carried out at least once a week. In some embodiments, the present invention is carried out substantially daily. In some embodiments, the method is carried out by a clinician. In other embodiments, the method is carried out by the patient.

The present inventors have further noticed that it has been recently reported that the cribriform plate represents one of the major avenues of CSF drainage from the brain. For example, Mollanji, Am. J. Physiol. Regulatory Intergrative Comp. Physiol. 282: R1593-R1599 (2002) studied the effect of blocking CSF absorption through the cribriform plate, reported finding increased resting intracranial pressures, and concluded that the olfactory pathway represents a major site of CSF drainage. Silver, Neuropathol. Appl. Neurobiol. 2002, February 28(1) 67-74 reports that recent studies in sheep suggest that a significiant proportion of global CSF drainage (50% or greater) occurs through the cribriform plate into nasal mucosal lymphatics. Zakharov, Microvascular Research, 67, (2004) 96-104, reports that, at baseline pressures, the majority of CSF clearance occurs through the cribriform plate.

It has further been reported that there is no conventional blood brain area in the area of the cribriform plate. Moran. J. Neurocytol 11, 721-46, 1982.

Accordingly, it is further believed that therapeutic access to the brain through the cribriform plate can be achieved because of physical imperfections in the dura and blood brain barrier near the cribriform plate that presently allow the drainage of CSF under pressure. In short, the present inventors believe that simply reversing the pressure gradient across the cribriform plate will allow for the introduction of therapeutic molecules from the nasal cavity through the cribriform plate and into the CSF and brain tissue.

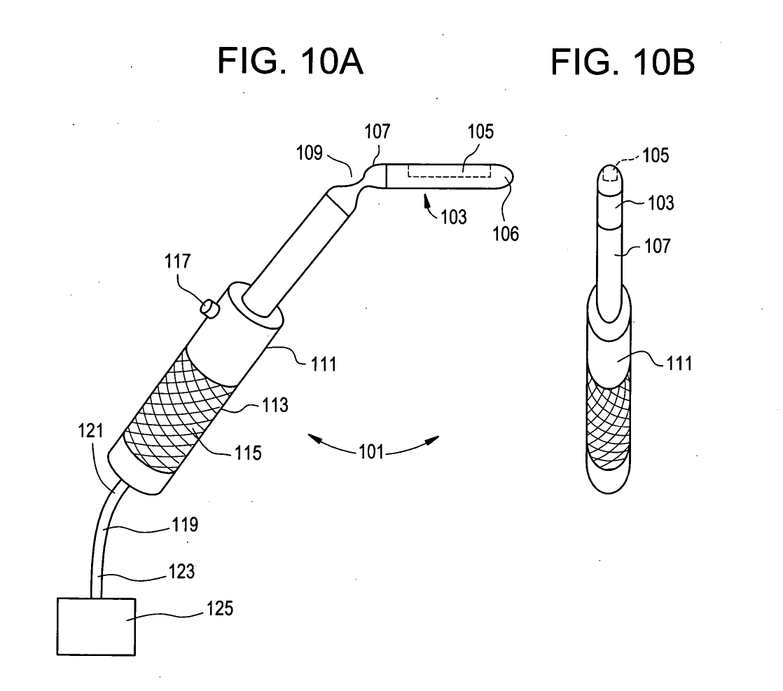

In order to accomplish this task, in some embodiments, and now referring to FIG. 10, there is provided a device 101 adapted for placement against the cribriform plate, comprising:
a) a distal portion 103 adapted to fit within an upper portion of a nasal cavity, the distal portion having a flexible outer portion (or plug) 106 and having an inner reservoir 105 oriented towards the cribriform plate,
b) a flexible intermediate portion 107 having an angled, narrowed portion 109,
c) a proximal portion 111 having a handgrip 113 having a knurled surface 115 and a pressure activation button 117,
d) a pump disposed within the proximal portion (not shown) and in fluid connection with the reservoir,
e) a drug delivery tube 119 having a proximal end 121 in fluid connection with the pump and a distal end 123, and
f) a container 125 containing a therapeutic agent and being in fluid connection with the distal end of the drug delivery tube.

Because delivery of the therapeutic relies upon mainteneance of the reverse pressure gradient at the cribriform plate, in some embodiments, the plug is made of a somewhat malleable material, such as rubber, that can be press fit into the space between the medial septum wall and the wall of the upper conchae, thereby providing a water-tight fit.

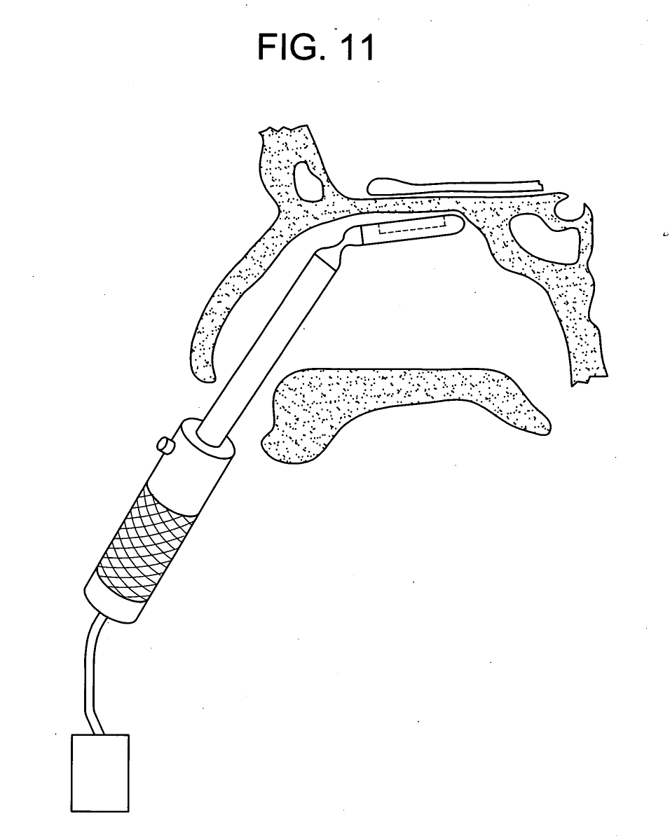

FIG. 11 shows the pressure-based delivery device of the present invention adjacent the cribriform plate.

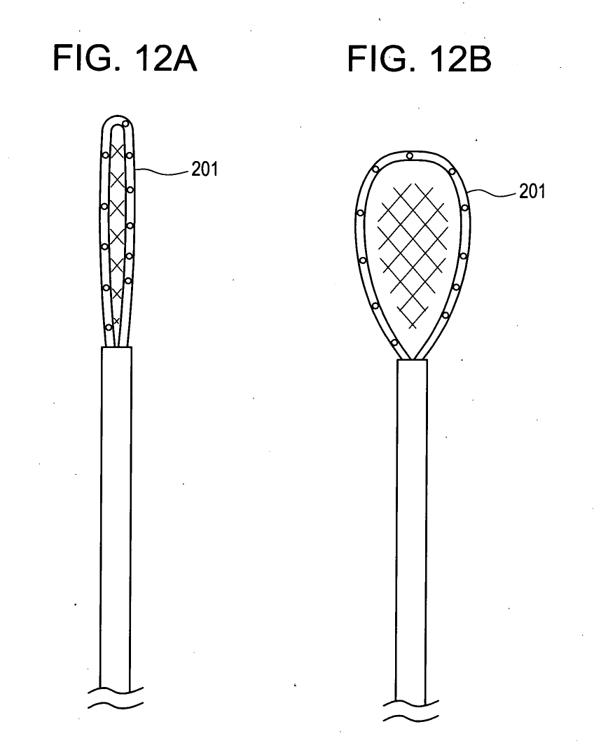

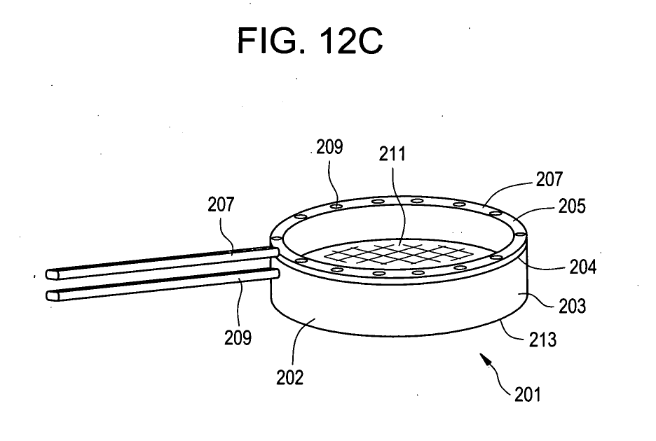

Now referring to FIGS. 12a-c, there is provided another embodiment suitable for pressurized delivery through the cribriform plate, wherein the distal portion of the device is controllably expandable. When such a device is used, the distal portion 201 of the device may travel up the nasal cavity in a substantially closed fashion (as in FIG. 12a), thereby reducing tissue, irritation and then expand (as in FIG. 12b) when it is pressed against the cribriform plate. This expandable embodiment is particularly useful for this application because the upper end of the nasal cavity adjacent the cribriform plate is often somewhat wider than the lower portions. Thus, the device will conform to the available space, while still providing a snug fit.

The expansion may be accomplished by any known means, including making the distal portion from a memory metal 203 that changes shape to the expanded shape upon heating. In other embodiments (not shown), the distal portion can have dimensions that provide to the distal portion an inherent spring portion. In other embodiments (not shown), the distal portion can comprise a balloon that can travel up the nasal cavity in a collapsed form and then expand once the cribriform plate is reach.

Now referring to FIG. 12c, there. is provided an intranasal device for delivering therapeutics through the cribriform late, comprising:
a) an expandable annulus 203 forming a reservoir, the annulus having an upper surface 204, a lower surface 213 and a proximal end portion 202 having a throughhole,
b) a flexible sheet 211 attached to the lower surface of the annulus,
c) a tubular member 205 located upon the upper surface of the expandable annulus, the tubular member having an upper surface 207 having a plurality of suction holes 209 thereon and a proximal portion 210 having a throughhole,
d) a delivery tube 209 connected to the throughhole of the annulus and adapted to deliver a therapeutic agent to the reservoir under pressure, and
e) a suction tube 207 connected to the throughhole of the tubular member and adapted to provide suction to the suction holes.

In some intranasal pressure-related embodiments, it may be helpful to reduce intracranial pressure in order to augmented the flow of the therapeutic agent across the cribriform plate. In such cases, it is preferred that a pressure-reducing agent (such as mannitol) be administered, or that a lumbar puncture be performed.

Although some aspects of the present invention is preferably directed to the use of selected molecules, such as reduced water, αMSH and lubeluzole, it is appreciated that novel devices for delivering therapeutics to the brain have also been disclosed and that these devices can be used in conjunction with many other therapeutic compounds. Therefore, the term “therapeutic agent” as defined herein, is an agent, or its pharmaceutically acceptable salt, or mixture of compounds, which has therapeutic, prophylactic, pharmacological, physiological or diagnostic effects on a mammal and may also include one compound or mixture of compounds that produce more than one of these effects. Suitable therapeutic, pharmacological, physiological and/or prophylactic biologically active agents can be selected from the following listed, and are given as examples and without limitation: amino acids, anabolics, analgesics and antagonists, anaesthetics, anti-adrenergic agents, anti-asthmatics, anti-atherosclerotics, antibacterials, anticholesterolics, anti-coagulants, antidepressants, antidotes, anti-emetics, anti-epileptic drugs, anti-fibrinolytics, anti-inflammatory agents, antihypertensives, antimetabolites, antimigraine agents, antimycotics, antinauseants, antineoplastics, anti-obesity agents, anti-Parkinson agents, antiprotozoals, antipsychotics, antirheumatics, antiseptics, antivertigo agents, antivirals, appetite stimulants, bacterial vaccines, bioflavonoids, calcium channel blockers, capillary stabilizing agents, coagulants, corticosteroids, detoxifying agents for cytostatic treatment, diagnostic agents (like contrast media, radiopaque agents and radioisotopes), drugs for treatment of chronic alcoholism, electrolytes, enzymes, enzyme inhibitors, ferments, ferment inhibitors, gangliosides and ganglioside derivatives, hemostatics, hormones, hormone antagonists, hypnotics, immunomodulators, immunostimulants, immunosuppressants, minerals, muscle relaxants, neuromodulators, neurotransmitters and nootropics, osmotic diuretics, parasympatholytics, para-sympathomimetics, peptides, proteins, psychostimulants, respiratory stimulants, sedatives, serum lipid reducing agents, smooth muscle relaxants, sympatholytics, sympathomimetics, vasodilators, vasoprotectives, vectors for gene therapy, viral vaccines, viruses, vitamins, oligonucleotides and derivatives, and any therapeutic agent capable of affecting the nervous system.

Modifications of the therapeutic agent and its functional fragments that either enhance or do not greatly affect the therapeutic nature of the agent are also included within the term “therapeutic agent.” Such modifications include, for example, additions, deletions or replacements of one or more amino acids from the native amino acid sequence of an enzyme agent with a structurally or chemically similar amino acid or amino acid analog. These modifications will either enhance or not significantly alter the structure, conformation or functional activity of the agent or a functional fragment thereof. Modifications that do not greatly affect the activity of the agent or its functional fragments can also include the addition or removal of sugar, phosphate or lipid groups as well as other chemical derivations known in the art. Additionally, an agent or its functional fragments can be modified by the addition of epitope tags or other sequences that aid in its purification and which do not greatly affect its activity. As used herein, the term “functional fragment,” in connection with a therapeutic agent, is intended to mean a portion of the agent that maintains the ability of the agent to delivery the intended therapy. A functional fragment can be, for example, from about 6 to about 300 amino acids in length, for example, from about 7 to about 150 amino acids in length, more preferably from about 8 to about 50 amino acids in length. If desired, a functional fragment can include regions of the agent with activities that beneficially cooperate with the ability to deliver the therapy. For example, a functional fragment of the therapeutic agent can include sequences that promote the ingrowth of cells, such as endothelial cells and macrophages, at the site of inflammation.

I claim:

1. A method of treating a patient having a disease of the central nervous system, comprising:
a) intranasally administering through a cribriform plate an effective amount of a therapeutic agent selected from the group consisting of reduced water, lubeluzole, catalase, SOD, melatonin, αMSH and Vitamins C and E.

2. The method of claim 1 wherein the intranasal administration is augmented by iontophoresis.

3. The method of claim 2 wherein the iontophoresis is accomplished with an intranasal probe having a reservoir of the therapeutic agent.

4. The method of claim 3 wherein the intranasal probe comprises a monopolar probe.

5. The method of claim 4 wherein the iontophoresis is accomplished with an anode placed upon a back of a head of the patient.

6. The method of claim 3 wherein the intranasal probe comprises a bipolar probe.

7. The method of claim 2 wherein the intranasal administration is accomplished by providing a pressure gradient across the cribriform plate.

8. The method of claim 7 wherein intracranial pressure within a skull of the patient is reduced.

9. The method of claim 1 wherein the therapeutic agent is reduced water.

10. The method of claim 1 wherein the therapeutic agent is lubeluzole.

11. A device for treating a patient having a disease of the central nervous system, comprising:
a) an intranasal container having an open distal end and a closed proximal end, the container forming a reservoir, and
b) a therapeutic agent selected from the group consisting of reduced water and lubeluzole contained within the reservoir.

12. The device of claim 11 wherein the therapeutic agent is reduced water having a redox potential of between −50 mV and −1000 mV.

13. The device of claim 11 wherein the device further comprises:
c) an electrode.

14. The device of claim 13 wherein the closed proximal end of the container forms an inner proximal face, and the electrode is attached to the inner proximal face of the container.

15. The device of claim 13 wherein the intranasal probe comprises a monopolar cathodic probe.

16. The device of claim 13 wherein the intranasal probe comprises a bipolar probe further comprising d) a first anode.

17. The device of claim 16 wherein the first anode is located distal to the cathode.

18. The device of claim 13 wherein the intranasal probe comprises a bipolar probe further comprising e) a second anode.

19. The device of claim 18 wherein the second anode is located proximal to the cathode.

20. The method of claim 11 wherein the container is pressurized.

21. The device of claim 11 further comprising:
c) a porous fibrous plug attached to the open distal end of the container.

22. The device of claim 11 wherein the therapeutic agent is lubeluzole.

23. A method of delivering charged molecules to a patient having a skull, a dura, and a subarachnoid space, comprising:
a) removing a portion of the skull to expose the dura, and
b) iontophoretically delivering a therapeutic agent through the exposed dura and into the subarachnoid space.

24. The method of claim 23 wherein the patient has suffered a stroke.

25. The method of claim 23 wherein the therapeutic agent is reduced water.

26. The method of claim 23 wherein the therapeutic agent is lubeluzole.

27. The method of claim 23 wherein step a) produces a hole in the skull, and further comprising the step of:
c) inserting a device comprising an electrode into the hole in the skull.

28. The device of claim 27 wherein the device has a threaded outer surface.

29. The device of claim 27 wherein the device has an inner surface defining a throughbore.

30. A method of treating a patient having a disease of the central nervous system, comprising:
a) intranasally administering through a cribriform plate an effective amount of a therapeutic agent, wherein the intranasal administration is accomplished by providing a pressure gradient across the cribriform plate.

31. The method of claim 30 wherein intracranial pressure is reduced.
